(19) United States
(12) Patent Application Publication
(10) Pub. No.: US 2025/0011347 A1
(43) Pub. Date: Jan. 9, 2025

(54) NOVEL PLASMALOGEN DERIVATIVE

(71) Applicants: Institute of Rheological Function of Food Co., Ltd., Fukuoka (JP); KYUSHU UNIVERSITY, NATIONAL UNIVERSITY CORPORATION, Fukuoka (JP)

(72) Inventors: Takehiko FUJINO, Fukuoka (JP); Shiro MAWATARI, Fukuoka (JP); Tatsuo OKAUCHI, Fukuoka (JP); Takuya HISAMITSU, Fukuoka (JP); Shamim NIWASE, Fukuoka (JP); Masanori HONSHO, Fukuoka (JP); Kinichi NAKASHIMA, Fukuoka (JP); Hideyuki NAKASHIMA, Fukuoka (JP)

(73) Assignees: Institute of Rheological Function of Food Co., Ltd., Fukuoka (JP); KYUSHU UNIVERSITY, NATIONAL UNIVERSITY CORPORATION, Fukuoka (JP)

(21) Appl. No.: 18/685,412

(22) PCT Filed: Aug. 22, 2022
(86) PCT No.: PCT/JP2022/031555

§ 371 Date: Feb. 21, 2024

(30) Foreign Application Priority Data
Aug. 23, 2021 (JP) 2021-135911

(51) Int. Cl. C07F 9/10; A61K 31/685; A61P 25/00; A61P 29/00

(52) U.S. Cl. CPC C07F 9/10; A61K 31/685; A61P 25/00; A61P 29/00

(57) ABSTRACT

A compound represented by formula (I), its racemic form, or their salt, wherein X represents an oxygen atom, a nitrogen atom or a sulfur atom; R¹ represents an unsaturated aliphatic hydrocarbon group; R² represents a saturated or unsaturated aliphatic hydrocarbon group; and R³ represents choline, ethanolamine, inositol or serine.

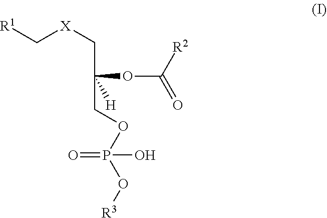

### TECHNICAL FIELD

The present invention relates to a novel compound (plasmalogen derivative) having a structure similar to a plasmalogen.

### BACKGROUND ART

While phospholipids are important as structural components of a biomembrane, approximately 18% of phospholipids of a mammal biomembrane are plasmalogens that are ether phospholipids. In particular, it is known that many of them are found in brain nerves, heart muscles, skeletal muscles, white blood cells and sperms.

Many of plasmalogens are bound to polyunsaturated fatty acids such as docosahexaenoic acids and arachidonic acids, etc. Therefore, they play not only a role as storage of second messengers for signals between cells such as prostaglandin and leukotriene, etc., generated from these polyunsaturated fatty acids, but also significant roles as cell fusion, ion transport, etc.

In addition, it has been made clear that plasmalogens themselves involve in signal transmission via a particular G-protein-coupled type receptors (GPCR). For example, while plasmalogens suppress neuronal cell death by reinforcing activity of protein phosphoenzyme such as AKT and ERK, etc. of a nerve cell (see Non-patent Literature 1), it was reported that 5 certain kinds of GPCR involve as a mechanism for such a cell signal transmission (see Non-patent Literature 2).

Further, when Lipopolysaccharide (LPS) was injected into an abdominal cavity of a mouse for seven days, IL-1β and TNF-α mRNA expressed strongly in its prefrontal cortex and hippocampus, and β amyloid (Aβ1-16) positive neurons expressed along with activation of glia cells. When plasmalogens were co-administered into the abdominal cavity after the LPS injection, they significantly reduced activation of glia cells involving cytokine production, so did accumulation of Aβ protein. In addition, although the contained amount of plasmalogens was reduced by LPS in the prefrontal area and hippocampus, such a reduction was suppressed by the co-administration of plasmalogens.

In other words, it is considered that plasmalogens have anti-neuroinflammatory and anti-amyloidogenic effects, thereby suggesting their preventive or improvement (therapeutic) application against Alzheimer's disease (see Non-patent Literature 3).

It is known that plasmalogens are decreased in patients with brain diseases such as dementia, Parkinson disease, depression and schizophrenia, diabetes, metabolic syndrome, ischemic heart disease, various infectious diseases, and immune disorder.

For example, it was reported in 1999 that in brains of patients with Alzheimer's disease (from dead bodies' brains), ethanolamine-type plasmalogens were quite significantly decreased in its prefrontal cortex and hippocampus (see Non-patent Literature 4). In addition, in 2007, it was reported that plasmalogens were decreased in serum of a patient suffering Alzheimer's disease (see Non-patent Literature 5).

Further, it was reported that choline-type plasmalogens were decreased in a group of ischemic heart disease patients as compared to those in a group of normal control (see Non-patent Literature 6).

Since it is considered that supplementing those decreased plasmalogens externally can expect preventing and improving effects of those diseases, various attempts have been made conventionally to extract those plasmalogens from an animal tissue. For example in Patent Literature 1, it is proposed a method of providing extraction processing to a chicken breast layer using ethanol as extraction solvent to collect an extraction liquid.

In addition, Patent Literature 2 proposes a method characterized in providing extraction processing to a bivalve such as scallops using stirred solvent of non-polar organic solvent and branched alcohol, followed by processing with phospholipase A1 (PLA1), to remove foreign substance of diacyl phospholipids by hydrolysis.

In addition, it was reported that a randomized double-blinded clinical trial was carried out to human patients with mild Alzheimer's disease or mild dementia where plasmalogens extracted from the above scallop strips were orally administered, as a consequence of which it strongly suggested that a cognitive function is improved for patients with mild Alzheimer's disease (see Non-patent Literature 7).

On the other hand, as a synthesis example of plasmalogen derivative, it is proposed a new plasmalogen precursor that couples α-lipoic acid at a sn-3 position of plasmalogens, for the purpose of preventing or improving various diseases caused from deficient plasmalogens by increasing decreased plasmalogen level (see Patent Literature 3). It was reported that such derivative was effective for a monkey model with Parkinson disease (see Non-patent Literature 8).

In addition, it is proposed in Patent Document 4 that a derivative with a sn-1 position acetylated is a good carrier of docosahexaenoic acid, which is effective for a mouse model with acute stroke (see Non-patent Literature 9).

### CITATION LIST

### Patent Literatures

Patent Literature 1: Japanese Patent No. 5483846
Patent Literature 2: Japanese Patent Application Laid-Open Publication No. 2016-108466
Patent Literature 3: International Publication No. 2010/071988
Patent Literature 4: International Publication No. 2013/037862

### Non-Patent Literatures

Non-Patent Literature 1: Md. Shamim Hossain et al, Plasmalogens rescue neuronal cell death through an activation of AKT and ERK survival signaling. PLoS ONE 8 (12): e83508, 2013
Non-Patent Literature 2: Md. Shamim Hossain et al, Neuronal Orphan G-Protein Coupled Receptor Proteins Mediate Plasmalogens-Induced Activation of ERK and Akt Signaling. PLoS ONE 11 (3):e0150846, 2016
Non-Patent Literature 3: M. Ifuku et al, Anti-inflammatory/anti-amyloidogenic effects of plasmalogens in lipopolysaccharide-induced neuroinflammation in adult mice. J of Neuroinflammation, 9:197, 2012
Non-Patent Literature 4: Z. Guan et al, Decrease and Structural Modifications of Phosphatidylethanolamine Plasmalogen in the Brain with Alzheimer Disease, J Neuropathol Exp Neurol, 58 (7), 7400-747, 1999
Non-Patent Literature 5: D. B. Goodenowe et al, Peripheral ethanolamine plasmalogen deficiency: a logical causative factor in Alzheimer's disease and dementia, J Lipid Res, 48, 2485-2498, 2007
Non-Patent Literature 6: Takeo SANADA et al, Serum Plasmalogens in Ischemic Heart Disease, J. Japan Atherosclerosis Society, 11, 535-539, 1983
Non-Patent Literature 7: T. Fujino et al, Efficacy and Blood Plasmalogen Changes by Oral Administration of Plasmalogen in Patients with Mild Alzheimer's Disease and Mild Cognitive Impairment: A Multicenter, Randomized, Double-blind, Placebo-controlled Trial, EBioMedicine, 17: 199-205, 2017
Non-Patent Literature 8: L. Gregoire et al, Plasmalogen precursor analog treatment reduces levodopa-induced dyskinesias in parkinsonian monkeys, Behav Brain Res, 286: 328-337, 2015
Non-Patent Literature 9: C. Fabien et al, Brain-Targeting Form of Docosahexaenoic Acid for Experimental Stroke Treatment: MRI Evaluation and Anti-Oxidant Impact, Current Neurovascular Res, 8 (2): 95-102, 2011
Non-Patent Literature 10: P. Wang et al, Improved Plasmalogen Synthesis Using Organobarium Intermediates, J. Org. Chem, 72: 5005-5007, 2007

### SUMMARY OF INVENTION

### Technical Problem

An object of the present invention is to provide a novel compound, having a structure similar to a plasmalogen, which exhibits an excellent anti-inflammatory effect.

### Solution to Problem

While conducting research on plasmalogens and similar compounds thereof, the present inventors have found that a plasmalogen derivative having an unsaturated bond at an sn-1 position exhibits an excellent anti-inflammatory effect even if a vinyl ether bond at the sn-1 position, which is a major feature of a plasmalogen, is not formed, and have completed to the present invention.

Specifically, the present invention is as follows.

[1] A compound represented by the general formula (I) or its racemic form, or their salt:

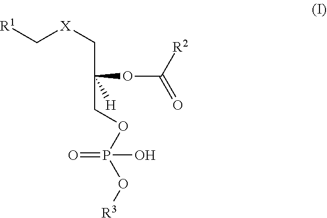

wherein X represents an oxygen atom, a nitrogen atom or a sulfur atom; R¹ represents an unsaturated aliphatic hydrocarbon group; R² represents a saturated or unsaturated aliphatic hydrocarbon group; and R³ represents choline, ethanolamine, inositol or serine.
   [2] The compound or its racemic form, or their salt according to [1], wherein X is an oxygen atom.
   [3] The compound or its racemic form, or their salt according to [1] or [2], wherein R¹ has at least one double bond.
   [4] The compound or its racemic form, or their salt according to [3], wherein R¹ has one double bond.
   [5] The compound its racemic form, or their salt according to [3] or [4], wherein the double bond of R¹ is present between the carbon at position 1 and the carbon at position 2, when the carbon bonded to the carbon bonded to X is defined as the carbon at position 1.
   [6] A composition comprising the compound, its racemic form, or their salt according to any one of [1] to [5] as an active ingredient.
   [7] The composition according to [6], wherein the composition has an anti-inflammatory effect.
   [8] The composition according to [6] or [7], wherein the composition is for preventing or ameliorating cranial nerve inflammatory diseases.
   [9] The composition according to [8], wherein the cranial nerve inflammatory diseases are at least one disease selected from dementia, Parkinson disease, depression and schizophrenia.
   [10] The composition according to [6] or [7], wherein the composition is for preventing or ameliorating Rett syndrome.
   [11] The composition according to any one of [6] to [9], wherein the composition is a pharmaceutical composition.

### Advantageous Effect of Invention

The novel compound of the present invention exhibits an excellent anti-inflammatory effect.

### BRIEF DESCRIPTION OF THE DRAWINGS

FIG. 1 shows the measurement results (semi-quantitative PCR) of the LPS-induced inflammatory cytokine IL-1β expression level (the relative expression level to the control) in microglial cells (BV2) of mice administered with the compound of the present invention (KIT-008).

FIG. 2 shows the measurement results (Western blotting) of the LPS-induced p65 nuclear expression level (the relative expression level to the control) in microglial cells (BV2) of mice administered with each of the compounds of the present invention (KIT-008 and KIT-020).

FIG. 3 shows the measurement results (Western blotting) of the LPS-induced p65 nuclear expression level (the relative expression level to the control) in microglial cells (MG6) of mice administered with each of the compounds of the present invention (KIT-019 and KIT-020).

FIG. 4 shows the measurement results (ELISA) of the LPS-induced inflammatory cytokine IL-1β expression level (the relative expression level to the control) in microglial cells (MG6) of mice administered with each of the compounds of the present invention (KIT-019 and KIT-020).

FIG. 5 shows the survival rate of the MeCP2-deficient mice administered with the compound of the present invention (KIT-008).

FIG. 6 shows the evaluation results of RTT-like symptoms of MeCP2-deficient mice administered with each of the compounds of the present invention (KIT-008, KIT-019 and KIT-020) by scoring.

FIG. 7 shows the change in body weight of MeCP2-deficient mice administered with each of the compounds of the present invention (KIT-008, KIT-019 and KIT-020).

FIG. 8 shows the change in body weight of wild-type mice administered with each of the compounds of the present invention (KIT-008, KIT-019 and KIT-020).

FIG. 9 shows photomicrographs showing that lysosomal acidification in neurons Neuro2a induced by the culture supernatant of BV2 microglia administered with LPS was impaired.

FIG. 10 shows photomicrographs showing that the impaired lysosomal acidification induced in neurons Neuro2a was suppressed by administration of each of the compounds of the present invention (KIT-008, KIT-019 and KIT-020).

FIG. 11 shows photomicrographs showing that accumulation of amyloid beta (Aβ) in lysosomes induced in neurons Neuro2a was suppressed by administration of the compound of the present invention (KIT-008).

FIG. 12 shows the results of the water maze test of mice with LPS-induced memory impairment administered with each of the compounds of the present invention (KIT-008 and KIT-020).

FIG. 13 shows s photomicrographs showing that LPS-induced amyloid beta (Aβ) deposition was suppressed in the mouse hippocampal tissue by administration of each of the compounds of the present invention (KIT-008 and KIT-020).

FIG. 14 shows a graph showing that LPS-induced amyloid beta (Aβ) deposition was suppressed in the mouse hippocampal tissue by administration of each of the compounds of the present invention (KIT-008 and KIT-020), the graph showing the average number of amyloid beta that deposited.

FIG. 15 shows photomicrographs showing that the increase in expression of inducible nitric oxide synthase (iNOS) was suppressed in the mouse hippocampal tissue by administration of each the compounds of the present invention (KIT-008 and KIT-020).

FIG. 16 shows a graph showing that the increase in expression of inducible nitric oxide synthase (iNOS) was suppressed in the mouse hippocampal tissue by administration of each of the compounds of the present invention (KIT-008 and KIT-020), the graph showing the average value of the number of iNOS-positive neurons.

FIG. 17 shows microphotographs showing that LPS-induced amoeboid shaped microglia (Iba1-positive cells) increase and amoeboid shaped astrocytes (GFAP-positive cells) increase were suppressed in the mouse hippocampal tissue by administration of each of the compounds of the present invention (KIT-008 and KIT-020).

FIG. 18 shows graphs showing that LPS-induced amoeboid shaped microglia (Iba1-positive cells) increase and amoeboid shaped astrocytes (GFAP-positive cells) increase and astrocytes (GFAP-positive cells) increase were suppressed in the mouse hippocampal tissue by administration of each of the compounds of the present invention (KIT-008 and KIT-020), the upper graphs showing the percentages of amoeboid shaped microglia and amoeboid shaped astrocytes and the lower graph showing the average value of the number of astrocytes (GFAP-positive cells).

### DESCRIPTION OF EMBODIMENTS

### [Novel Compound of the Present Invention]

The compound of the present invention is a compound represented by the following general formula (I) or its racemic form, or their salt. The carbon atom of the glycerol backbone of the compound represented by the general formula (I) may have a substituent. Examples of the substituent include an alkyl group having 1 to 4 carbon atoms and an alkoxy group having 1 to 4 carbon atoms.

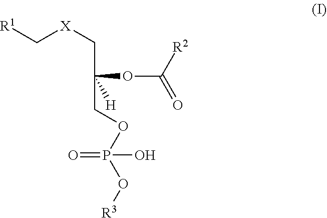

In the formula (I), X represents an oxygen atom, a nitrogen atom or a sulfur atom and is preferably an oxygen atom.

R¹ represents an unsaturated aliphatic hydrocarbon group and preferably one having at least one double bond. The double bond of R¹ may be present at any position, but is preferably at least between the carbon at position 1 and the carbon at position 2, when the carbon bonded to the carbon adjacent to X is defined as the carbon at position 1.

In addition, R¹ may be linear or branched, but is preferably linear. The number of carbon atoms in R¹ is preferably 4 to 50, more preferably 8 to 30, even more preferably 10 to 25, and particularly preferably 15 to 20. R¹ may have a substituent, and examples of the substituent include an alkoxy group having 1 to 4 carbon atoms.

R² represents a saturated or unsaturated aliphatic hydrocarbon group, and is preferably an unsaturated aliphatic hydrocarbon group. The unsaturated aliphatic hydrocarbon group of R² preferably has at least one double bond, and may have two or more double bonds.

R² may be linear or branched, but is preferably linear. The number of carbon atoms in R² is preferably 1 to 50, more preferably 8 to 30, even more preferably 10 to 25, and particularly preferably 15 to 20. R² may have a substituent, and examples of the substituent include an alkoxy group having 1 to 4 carbon atoms.

Specifically, when R² is considered as R²COOH, R²COOH preferably represents an ω-3 fatty acid, an ω-6 fatty acid, an ω-7 fatty acid, an ω-9 fatty acid or an ω-10 fatty acid; more preferably an ω-3 fatty acid, an ω-6 fatty acid or an ω-9 fatty acid; and particularly preferably an ω-6 fatty acid.

R³ represents choline, ethanolamine, inositol or serine. These may have as substituent, and examples of the substituent include an alkyl group having 1 to 4 carbon atoms and an alkoxy group having 1 to 4 carbon atoms.

The novel compound of the present invention has an anti-inflammatory effect. It is also effective in preventing or ameliorating (treating) Rett syndrome.

### [Composition of the Present Invention]

The composition of the present invention is characterized by comprising the novel compound of the present invention represented by the above general formula (I) or its racemic form, or their salt as an active ingredient. The composition of the invention may also comprise other pharmaceutically acceptable ingredients.

The composition of the present invention has an anti-inflammatory effect, and it can be therefore used for preventing or ameliorating (treating) inflammatory diseases, and is particularly effective in preventing or ameliorating (treating) cranial nerve inflammatory diseases. Specifically, it can be suitably used for preventing or ameliorating (treating) cranial nerve inflammatory diseases such as dementia, Parkinson disease, depression and schizophrenia, and is particularly effective against Alzheimer's dementia. It is also effective in improving brain function.

The composition of the present invention is also effective in preventing or ameliorating (treating) Rett syndrome, which is a progressive neurodevelopmental disorder caused by mutations in the MECP2 gene located on the X chromosome.

The composition of the present invention can be used as a pharmaceutical or health food. The composition of the present invention can be also used as an oral or parenteral agent.

When used as an oral agent, examples of the dosage forms thereof include a tablet, a capsule, a powder, a granule, a liquid, a granulate, a rod, a plate, a block, a solid, a pill, a paste, a cream, a caplet, a gel, a chewable and a stick.

Examples of the parenteral agent include an external preparation, or an injection and a drip.

### [Method for Producing the Novel Compound of the Present Invention]

The novel compound of the present invention can be obtained, for example, by reacting (R)-(2,2-dimethyl-1,3-dioxolan-4-yl)methanol represented by the following structural formula with a compound having R¹ to R³. The compound represented by the following structural formula may be one protected with a protecting group other than an acetonide.

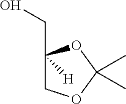

Here, examples of the compound having R¹ include a halogenated unsaturated aliphatic hydrocarbon (A-C—R¹ (A; halogen)). For example, when —R¹ is (—C═C—R^{1′}), the halogenated unsaturated aliphatic hydrocarbon (A-C—C═C—R^{1′}) can be produced as follows.

For example, an alcohol having a triple bond such as propargyl alcohol represented by the following chemical formula is used as a starting material.

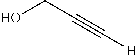

First, a protecting group (Z′) is introduced into the OH group of the alcohol having a triple bond.

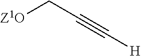

Subsequently, a desired aliphatic group (R^{1′}) is introduced by reacting the alcohol having the alcohol containing the protected triple bond with a halogenated aliphatic hydrocarbon. That is, in this step, the alcohol is reacted with the halogenated aliphatic hydrocarbon having the desired carbon chain, so that the carbon chain (R^{1′}) of the halogenated unsaturated aliphatic hydrocarbon (A-C—C═C—R^{1′}) finally produced can be a desired one.

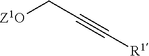

Then, the protecting group (Z¹) introduced above is deprotected.

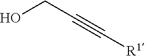

Subsequently, the resulting compound is converted into an alcohol having a cis-double bond (HO—C—C═C—R1′).

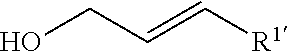

Finally, a halogen is introduced to convert the resulting compound into a halogenated unsaturated aliphatic hydrocarbon (A-C—C═C—R^{1′} (A; halogen)).

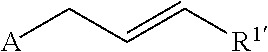

Here, examples of the halogen (A) include chlorine, bromine and iodine. When chlorine is introduced as a halogen, it can be introduced using PCl₃, SOCl₂, (COCl)₂, a combination of CCl₄ and PPh₃, or a combination of N-chlorosuccinimide (NCS) and PPh₃, or the like. When bromine is introduced as a halogen, it can be introduced using PBr₃, a combination of CBr₄ and PPh₃, a combination of N-bromosuccinimide (NBS) and PPh₃, or a combination of imidazole, PPh₃ and Br₂, or the like. When iodine is introduced as a halogen, it can be introduced using a combination of imidazole, PPh₃ and 12, or a combination of N-iodosuccinimide (NIS) and PPh₃, or the like.

The novel compound of the present invention is produced using the halogenated unsaturated aliphatic hydrocarbon (A-C—C═C—R^{1′} (A-C—R¹)) produced above.

First, this halogenated unsaturated aliphatic hydrocarbon (A-C—R¹) is reacted with (R)-(2,2-dimethyl-1,3-dioxolan-4-yl)methanol to obtain the following compound.

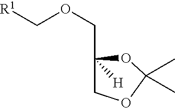

Subsequently, the acetonide of the above compound is deprotected to obtain the following compound.

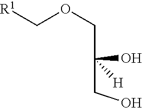

Subsequently, protecting groups (Z, Z′) are introduced into the two OH groups.

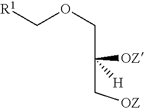

Thereafter, the protecting group (Z) is selectively deprotected to provide an OH group.

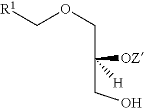

Then, a substituent containing a phosphoric group and R³ is introduced to obtain the following compound. The phosphoric group and R³ each protected with a protecting group is preferably used.

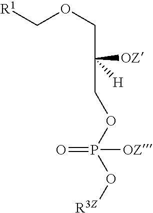

Subsequently, the protecting group (Z′) is deprotected to provide an OH group.

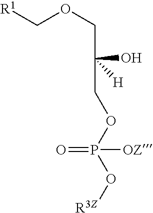

Thereafter, the deprotected OH group is condensed with R²COOH.

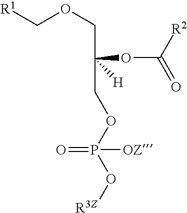

Finally, the protecting groups of the phosphoric group and R^{3Z} are subjected to deprotection or the like to obtain the following compound of the present invention.

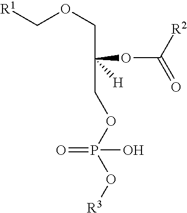

### Example 1

Compounds of the present invention represented by the following structural formulas, KIT-008, KIT-019 and KIT-020 are produced. The summary is shown below.

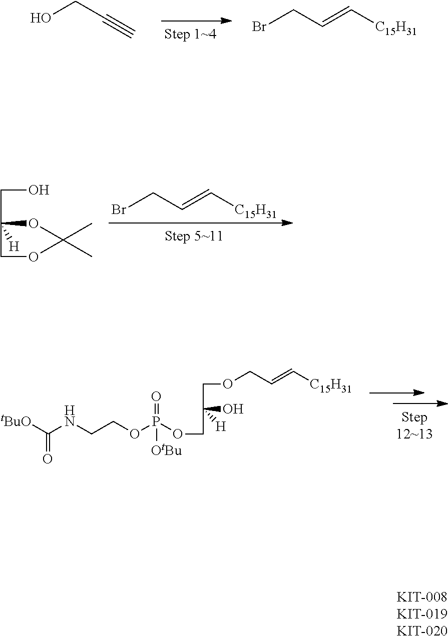

Hereinafter, this Example will be specifically described.

### <Production of KIT-008>

### Step 1: Protection of OH Group of Propargyl Alcohol

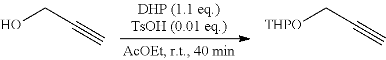

p-toluenesulfonic acid monohydrate (190 mg, 1.0 mmol) was added to a solution of propargyl alcohol (5.6 g, 100 mmol) in ethyl acetate (100 mL) at room temperature. 3,4-Dihydro-2H-pyran (10 mL, 110 mmol) was slowly added thereto at 0° C. This solution was stirred at 0° C. for 5 minutes, and the reaction mixture was then allowed to warm to room temperature. After the reaction mixture was stirred at room temperature for 40 minutes, a saturated NaHCO₃ was added thereto and extracted twice with ethyl acetate. The ethyl acetate layers were combined, washed with water and a saturated brine and then dried over Na₂SO₄, and the solvent was distilled off under reduced pressure. The residue was purified by distillation under reduced pressure (boiling point 76° C./10 mmHg) to obtain a THP-protected propargyl alcohol as a yellow liquid (11.9 g, 85%).

¹H NMR (500 MHz, CDCl₃) δ 4.83 (1H, d, J=3.4 Hz), 4.30 (1H, dd, J=2.4, 15.7 Hz), 4.24 (1H, dd, J=2.4, 15.7 Hz), 3.84 (1H, ddd, J=2.8, 9.0, 11.5 Hz), 3.84 (1H, ddt, J_{d}=1.5, 11.1 Hz, Jₜ=4.3 Hz), 2.42 (1H, t, J=2.4 Hz), 1.89-1.79 (1H, m), 1.78-1.71 (1H, m), 1.67-1.50 (4H, m).

### Step 2: Introduction of Aliphatic Group

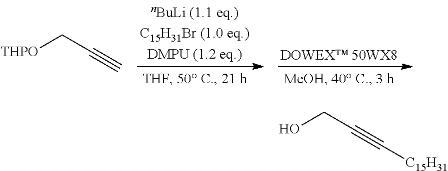

To a solution of the THP-protected propargyl alcohol (4.6 g, 33 mmol) in tetrahydrofuran (122 mL) was added dropwise 21 mL (33 mmol) a solution (1.57 mol/L) of ⁿBuLi in hexane at −78° C. 1-bromopentadecane (8.7 g, 30 mmol) was slowly added at 0° C., allowed to warm to room temperature, and then heated at 50° C. for 21 hours. The mixture was allowed to cool to room temperature, and a saturated aqueous NH₄Cl solution was added thereto and extracted twice with Et₂O. The Et₂O layers were combined, washed with a saturated brine, then dried over Na₂SO₄, and filtered through a pad of silica gel. The filtrate was concentrated under reduced pressure. The residue was dissolved in methanol (73 mL), DOWEX™ 50WX8 (a strongly acidic ion exchange resin) (994 mg) was added thereto, and the mixture was stirred at 40° C. for 3 hours. The reaction mixture was filtered and the filtrate was concentrated under reduced pressure. The residue was dissolved in petroleum ether at 30 to 40° C. and the resulting solution was allowed to cool to room temperature. The solution was cooled to −78° C. and the resulting precipitate was collected by filtration to obtain the desired alcohol as a white solid (6.1 g, 77%).

¹H NMR (500 MHz, CDCl₃) δ 4.25 (2H, t, J=2.1 Hz), 2.21 (2H, tt, J=2.1, 7.1 Hz), 1.60 (1H, brs), 1.50 (2H, tt, J=7.4, 7.4 Hz), 1.40-1.33 (2H, m), 1.33-1.21 (22H, m), 0.88 (3H, t, J=7.0 Hz).

### Step 3: Conversion to Cis-Allyl Alcohol

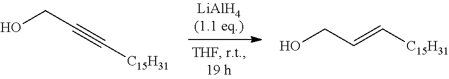

A solution (4 mL) of octadec-2-yn-1-ol (266 mg, 1.0 mmol) in tetrahydrofuran was added to a solution (6 mL) of LiAlH₄ (42 mg, 1.1 mmol) in tetrahydrofuran at 0° C., allowed to warm to room temperature and stirred for 19 hours. A saturated aqueous solution of Rochelle salt was added to the reaction mixture at 0° C., stirred for 1 hour, and then filtered. The filtrate was extracted twice with ethyl acetate. The ethyl acetate layers were combined, washed with water and a saturated brine and then dried over Na₂SO₄, and the solvent was distilled off under reduced pressure. The residue was purified by column chromatography to obtain the desired (E)-octadec-2-en-1-ol (244 mg, 91%) as a white solid.

¹H NMR (500 MHz, CDCl₃) δ 5.73-5.60 (2H, m), 4.11-4.07 (2H, m), 2.06-2.01 (2H, m), 1.40-1.34 (2H, m), 1.32-1.21 (24H, m), 0.88 (3H, t, J=7.0 Hz).

### Step 4: Halogenation of Alcohol

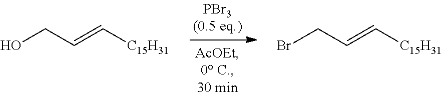

PBr₃ was added to a solution of (E)-octadec-2-en-1-ol in ethyl acetate at 0° C., and was stirred as it was for 30 minutes. Ice water was added thereto and extracted twice with ethyl acetate. The ethyl acetate layers were combined, washed with a saturated brine and then dried over Na₂SO₄, and the solvent was distilled off under reduced pressure. The residue was purified by column chromatography to obtain the desired (E)-1-bromooctadec-2-ene as a colorless liquid.

¹H NMR (500 MHz, CDCl₃) δ 5.78 (1H, dt, J_{d}=15.1 Hz, Jₜ=6.6 Hz), 5.68 (1H, dtt, J_{d}=15.1 Hz, Jₜ=1.2, 7.5 Hz), 3.95 (2H, dd, J=0.6, 7.5 Hz), 2.05 (2H, dt, J_{d}=7.0 Hz, Jₜ=7.0 Hz), 1.40-1.34 (2H, m), 1.32-1.21 (24H, m), 0.88 (3H, t, J=7.0 Hz).

### Step 5: Introduction of aliphatic group at Sn-1 position of (R)-(2,2-dimethyl-1,3-dioxolan-4-yl)methanol (hereinafter referred to as a substrate)

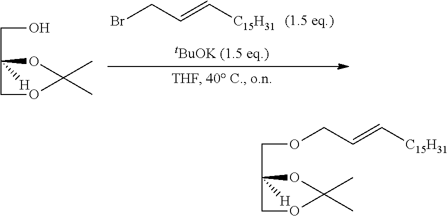

(R)-(2,2-dimethyl-1,3-dioxolan-4-yl)methanol (1.30 g, 9.8 mmol) was added to a solution (50 mL) of ^{t}BuOK (1.65 g, 14.7 mmol) in tetrahydrofuran at 0° C., and the mixture was stirred as it was for 1 hour. A solution (50 mL) of (E)-1-bromooctadec-2-ene (4.86 g, 14.7 mmol) prepared above in tetrahydrofuran was added thereto, heated to 40° C., and was stirred as it was overnight. A phosphate buffer (pH 7) was added thereto, and extracted twice with Et₂O. The Et₂O layers were combined, washed with water and a saturated brine and then dried over Na₂SO₄, and the solvent was distilled off under reduced pressure. The residue was purified by silica gel column chromatography to obtain the desired (R,E)-2,2-dimethyl-4-((octadec-2-en-1-yloxy)methyl)-1,3-dioxolane as a colorless liquid (3.62 g, 97%).

¹H NMR (500 MHz, CDCl₃) δ 5.69 (1H, dt, J_{d}=15.3 Hz, Jₜ=6.7 Hz), 5.53 (1H, dtt, J_{d}=15.3 Hz, Jₜ=1.4, 6.3 Hz), 4.28 (1H, tt, J=6.0, 6.0 Hz), 4.06 (1H, dd, J=6.4, 8.2 Hz), 4.01-3.93 (2H, m), 3.72 (1H, dd, J=6.4, 8.3 Hz), 3.50 (1H, dd, J=5.9, 9.8 Hz), 3.41 (1H, dd, J=5.6, 9.8 Hz), 2.03 (2H, dt, J_{d}=6.9 Hz, Jₜ=6.9 Hz), 1.43 (3H, s), 1.39-1.33 (2H, m), 1.36 (3H, s), 1.32-1.21 (24H, m), 0.88 (3H, t, J=7.0 Hz).

### Step 6: Deprotection of Substrate

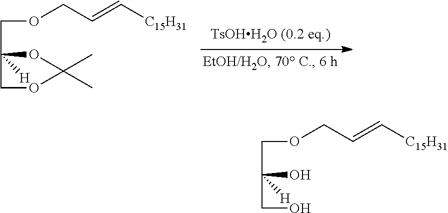

To a solution of (R,E)-2,2-dimethyl-4-((octadec-2-en-1-yloxy)methyl)-1,3-dioxolane (6.91 g, 18.0 mmol) in ethanol (60 mL) and water (7.5 mL) was added a solution (15 mL) of tosylic acid monohydrate (0.69 g, 3.6 mmol) in ethanol at 0° C. The mixture was heated to 70° C. and stirred as it was for 6 hours, then cooled to 0° C. and neutralized with triethylamine. The mixture was concentrated under reduced pressure. The residue was purified by column chromatography to obtain the desired (S,E)-3-(octadec-2-en-1-yloxy)propane-1,2-diol as a white solid (5.9 g, 95%).

¹H NMR (500 MHz, CDCl₃) δ 5.70 (1H, dt, J_{d}=15.3 Hz, Jₜ=6.8 Hz), 5.53 (1H, dtt, J_{d}=15.3 Hz, Jₜ=1.4, 6.3 Hz), 3.96 (2H, d, J=6.2 Hz), 3.90-3.85 (1H, m), 3.72 (1H, ddd, J=4.0, 7.0, 11.2 Hz), 3.65 (1H, dt, J_{d}=11.0 Hz, Jₜ=5.4 Hz), 3.54 (1H, dd, J=3.8, 9.7 Hz), 3.49 (1H, dd, J=6.3, 9.7 Hz), 2.56 (1H, d, J=5.0 Hz), 2.04 (2H, dt, J_{d}=6.8 Hz, Jₜ=6.9 Hz), 1.40-1.34 (2H, m), 1.32-1.21 (24H, m), 0.88 (3H, t, J=7.0 Hz).

### Step 7: Protection of OH Group at Sn-3 Position of Substrate

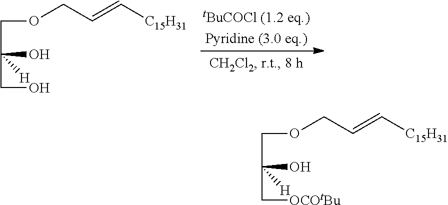

To a solution (13 mL) of (S,E)-3-(octadec-2-en-1-yloxy)propane-1,2-diol (436 mg, 1.27 mmol) and pyridine (0.31 mL, 3.81 mmol) in dichloromethane was added pivaloyl chloride (0.19 mL, 1.52 mmol) at 0° C. The reaction mixture was allowed to warm to room temperature and was stirred as it was for 8 hours. A phosphate buffer (pH 7) was added thereto, and extracted twice with Et₂O. The Et₂O layers were combined, washed with water and a saturated brine and then dried over Na₂SO₄, and the solvent was distilled off under reduced pressure. The residue was purified by column chromatography to obtain the desired (R,E)-2-hydroxy-3-(octadec-2-en-1-yloxy)propyl pivalate as a colorless liquid (440 mg, 81%).

¹H NMR (500 MHz, CDCl₃) δ 5.70 (1H, dt, J_{d}=15.3 Hz, Jₜ=6.7 Hz), 5.53 (1H, dtt, J_{d}=15.3 Hz, Jₜ=1.4, 6.3 Hz), 4.16 (1H, dd, J=4.8, 11.4 Hz), 4.13 (1H, dd, J=5.5, 11.3 Hz), 4.03-3.98 (1H, m), 3.96 (2H, d, J=6.3 Hz), 3.49 (1H, dd, J=4.3, 9.7 Hz), 3.43 (1H, dd, J=6.3, 9.7 Hz), 2.46 (1H, d, J=4.3 Hz), 2.04 (2H, dt, J_{d}=6.9 Hz, Jₜ=6.9 Hz), 1.40-1.34 (2H, m), 1.32-1.22 (24H, m), 1.22 (9H, s), 0.88 (3H, t, J=7.0 Hz).

### Step 8: Protection of OH Group at Sn-2 Position of Substrate

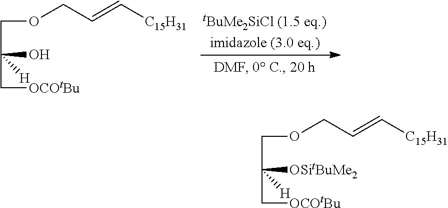

To a solution (9 mL) of tert-butyldimethylchlorosilane (R,E)-2-hydroxy-3-(octadec-2-en-1-yloxy)propyl pivalate (774 mg, 1.81 mmol) and imidazole (493 mg, 7.24 mmol) in DMF was added a solution (9 mL) of tert-butyldimethylchlorosilane (546 mg, 3.62 mmol) in DMF at 0° C. Thereafter, the mixture was allowed to warm to room temperature and stirred for 20 hours. A phosphate buffer (pH 7) was added thereto, and extracted twice with Et₂O. The Et₂O layers were combined, washed with water and a saturated brine and then dried over Na₂SO₄, and the solvent was distilled off under reduced pressure. The residue was purified by column chromatography to obtain the desired (R,E)-2-((tert-butyldimethylsilyl)oxy)-3-(octadec-2-en-1-yloxy)propyl pivalate as a colorless liquid. (921 mg, 94%).

¹H NMR (500 MHz, CDCl₃) δ 5.68 (1H, dt, Jₜ=15.3 Hz, Jₜ=6.7 Hz), 5.51 (1H, dt, J_{d}=15.3 Hz, Jₜ=6.2 Hz), 4.19-4.14 (1H, m), 4.02-3.95 (2H, m), 3.93 (2H, dd, J=0.6, 6.2 Hz), 3.49 (2H, dd, J=4.3, 9.7 Hz), 3.43-3.35 (2H, m), 2.03 (2H, dt, J_{d}=6.9 Hz, Jₜ=7.0 Hz), 1.40-1.32 (2H, m), 1.32-1.22 (24H, m), 1.20 (9H, s), 0.88 (3H, t, J=6.9 Hz), 0.88 (9H, s), 0.09 (6H, s).

### Step 9: Deprotection at Sn-3 Position of Substrate

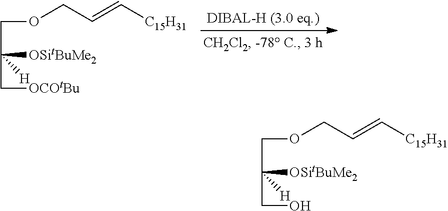

To a solution (60 mL) of (R,E)-2-((tert-butyldimethylsilyl)oxy)-3-(octadec-2-en-1-yloxy)propyl pivalate (915 mg, 1.69 mmol) in dichloromethane was added a 1.014 mol/L solution (5 mL, 5.07 mmol) of diisobutylaluminum hydride in toluene at −78° C. Thereafter, the mixture was stirred for 3 hours. A 30% aqueous solution of Rochelle salt was added thereto, stirred for 1 hour, filtered through Celite, and extracted twice with ethyl acetate. The ethyl acetate layers were combined, washed with water and a saturated brine and then dried over Na₂SO₄, and the solvent was distilled off under reduced pressure. The residue was purified by column chromatography to obtain the desired (S,E)-2-((tert-butyldimethylsilyl)oxy)-3-(octadec-2-en-1-yloxy)propan-1-ol as a colorless liquid (756 mg, 98%).

¹H NMR (500 MHz, CDCl₃) δ 5.68 (1H, dt, J=15.3 Hz, Jₜ=6.8 Hz), 5.51 (1H, dt, J_{d}=15.3 Hz, Jₜ=6.2 Hz), 3.92 (2H, d, J=6.2 Hz), 3.91-3.87 (1H, m), 3.65 (1H, dt, J_{d}=11.1 Hz, Jₜ=4.7 Hz), 3.58 (1H, ddd, J=4.5, 7.1, 11.3 Hz), 3.41 (2H, d, J=6.2 Hz), 2.12-2.08 (1H, m), 2.03 (2H, dt, J_{d}=7.0 Hz, Jₜ=7.0 Hz), 1.40-1.33 (2H, m), 1.32-1.21 (24H, m), 0.90 (9H, s), 0.88 (3H, t, J=7.1 Hz), 0.10 (3H, s), 0.09 (3H, s).

### Step 10: Introduction of Protected Phosphate Group and R³ at Sn-3 Position of Substrate

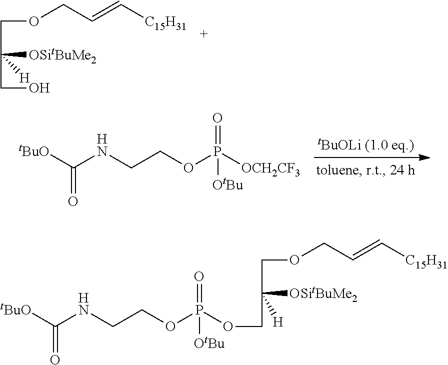

To a solution (8 mL) of (S,E)-2-((tert-butyldimethylsilyl)oxy)-3-(octadec-2-en-1-yloxy)propan-1-ol (722 mg, 1.58 mmol) in toluene was added a 0.1 mol/L solution (16 mL, 1.60 mmol) of ^{t}BuOLi in hexane at 0° C., and the mixture was stirred as it was for 1 hour. A solution (8 mL) of tert-butyl (2-((tert-butoxy(2,2,2-trifluoroethoxy)phosphoryl)oxy)ethyl)carbamate (899 mg, 2.37 mmol) in toluene was added thereto at 0° C., was then allowed to warm to room temperature, and was stirred as it was for 24 hours. A phosphate buffer (pH 7) was added thereto, and extracted twice with ethyl acetate. The ethyl acetate layers were combined, washed with water and a saturated brine and then dried over Na₂SO₄, and the solvent was distilled off under reduced pressure. The residue was purified by column chromatography to obtain the desired tert-butyl (2-((tert-butoxy((R)-2-((tert-butyldimethylsilyl)oxy)-3-(((E)-octadec-2-en-1-yl)oxy)propoxy)phosphoryl)oxy)ethyl)carbamate as a colorless liquid (1.035 g, 89%).

¹H NMR (500 MHz, CDCl₃) δ 5.71-5.64 (1H, m), 5.55-5.47 (1H, m), 5.15 (1H, brs), 4.09-4.02 (3H, m), 4.00-3.88 (4H, m), 3.44-3.35 (4H, m), 2.03 (2H, dt, J_{d}=6.5 Hz, Jₜ=6.6 Hz), 1.50 (9H, s), 1.44 (9H, s), 1.39-1.33 (2H, m), 1.32-1.22 (24H, m), 0.89 (9H, s), 0.88 (3H, t, J=7.1 Hz), 0.094+0.091+0.086+0.064+0.060 (6H, five singlet signals).

### Step 11: Deprotection of OH Group at Sn-2 Position of Substrate

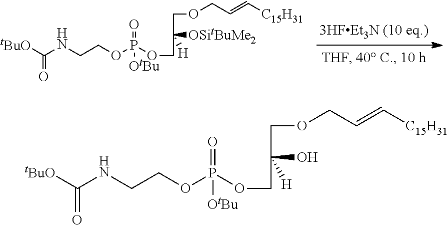

To a solution (14 mL) of tert-butyl (2-((tert-butoxy((R)-2-((tert-butyldimethylsilyl)oxy)-3-(((E)-octadec-2-en-1-yl)oxy)propoxy)phosphoryl)oxy)ethyl)carbamate (1.00 g, 1.36 mmol) in tetrahydrofuran was added triethylamine trihydrofluoride (1.7 mL, 13.6 mmol) at room temperature. Thereafter, the mixture was heated to 40° C. and was stirred as it was for 10 hours. A saturated aqueous sodium hydrogen carbonate solution was added thereto and extracted twice with ethyl acetate. The ethyl acetate layers were combined, washed with a saturated brine and then dried over Na₂SO₄, and the solvent was distilled off under reduced pressure. The residue was purified by column chromatography to obtain the desired tert-butyl (2-((tert-butoxy((R)-2-hydroxy-3-(((E)-octadec-2-en-1-yl)oxy)propoxy)phosphoryl)oxy)ethyl)carbamate as a colorless liquid (0.535 g, 65%).

¹H NMR (500 MHz, CDCl₃) δ 5.73-5.66 (1H, m), 5.55-5.48 (1H, m), 5.15 (1H, brs), 4.15-3.98 (5H, m), 3.96 (2H, dd, J=0.8, 6.3 Hz), 3.48 (2H, d, J=5.3 Hz), 3.43-3.38 (2H, m), 3.10 (1H, brs), 2.03 (2H, dt, J_{d}=7.1 Hz, J=7.1 Hz), 1.51 (9H, s), 1.44 (9H, s), 1.39-1.33 (2H, m), 1.32-1.22 (24H, m), 0.88 (3H, t, J=7.0 Hz).

### Step 12: Introduction of Aliphatic Group (R²) at Sn-2 Position of Substrate

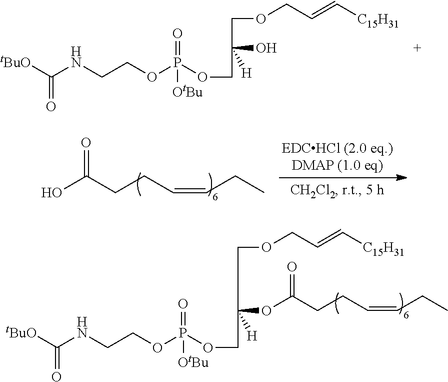

To a solution (0.5 mL) of tert-butyl (2-((tert-butoxy((R)-2-hydroxy-3-(((E)-octadec-2-en-1-yl)oxy)propoxy)phosphoryl)oxy)ethyl)carbamate (131.6 mg, 0.21 mmol) in dichloromethane was added a solution (0.5 mL) of 1-(3-dimethylaminopropyl)-3-ethylcarbodiimide hydrochloride (81.8 mg, 0.42 mmol) and dimethylaminopyridine (26.7 mg, 0.21 mmol) and docosahexaenoic acid (93.4 mg, 0.28 mmol) in dichloromethane at room temperature, and the mixture was stirred as it was for 5 hours. The reaction mixture was concentrated as it was under reduced pressure. The residue was purified by column chromatography to obtain the desired (2R)-1-((tert-butoxy(2-((tert-butoxycarbonyl)amino)ethoxy)phosphoryl)oxy)-3-(((E)-octadec-2-en-1-yl)oxy)propan-2-yl (4Z,7Z,10Z,13Z,16Z,19Z)-docosa-4,7,10,13,16,19-hexaenoate as a colorless liquid (0.535 g, 65%).

¹H NMR (500 MHz, CDCl₃) δ 5.72-5.65 (1H, m), 5.53-5.46 (1H, m), 5.43-5.28 (12H, m), 5.19-5.12 (2H, m), 4.25-4.10 (2H, m), 4.08-4.02 (2H, m), 3.97-3.90 (2H, m), 3.59 (2H, d, J=5.2 Hz, one diastereomer), 3.55 (2H, d, J=5.2 Hz, the other diastereomer), 3.42-3.37 (2H, m), 2.87-2.80 (10H, m), 2.41-2.39 (4H, m), 2.11-2.00 (4H, m), 1.50 (9H, s, the other diastereomer), 1.49 (9H, s, one diastereomer), 1.44 (9H, s), 1.39-1.33 (2H, m), 1.32-1.22 (24H, m), 0.97 (3H, t, J=7.5 Hz), 0.89 (3H, t, J=7.0 Hz).

### Step 13: Deprotection and Amination at Sn-3 Position of Substrate

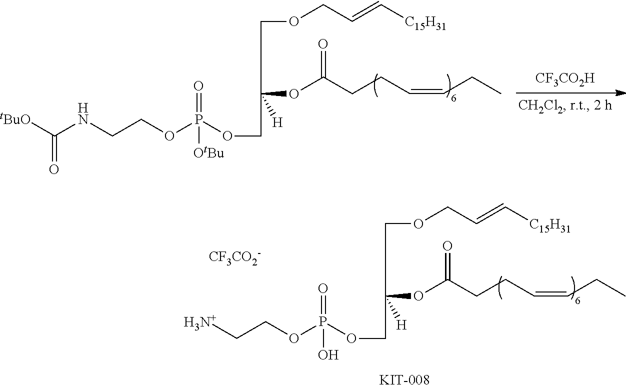

To a solution (0.3 mL) of (2R)-1-((tert-butoxy(2-((tert-butoxycarbonyl)amino)ethoxy)phosphoryl)oxy)-3-(((E)-octadec-2-en-1-yl)oxy)propan-2-yl(4Z,7Z,10Z,13Z,16Z,19Z)-docosa-4,7,10,13,16,19-hexaenoate (29.1 mg, 0.031 mmol) in dichloromethane was added 0.3 mL of trifluoroacetic acid at room temperature, and the mixture was stirred as it was for 2 hours. The reaction mixture was concentrated as it was under reduced pressure to obtain the desired 2-((((R)-2-(((4Z,7Z,10Z,13Z,16Z,19Z)-docosa-4,7,10,13,16,19-hexenoyl)oxy)-3-(((E)-octadec-2-en-1-yl)oxy)propoxy)(hydroxy)phosphoryl)oxy)ethane-1-ammonium 2,2,2-trifluoroacetate (KIT-008) as a colorless liquid (27.4 mg, quant).

¹H NMR (500 MHz, CDCl₃) δ 8.09 (3H, brs), 5.67 (1H, dt, J_{d}=15.1 Hz, Jₜ=6.9 Hz), 5.48 (1H, dt, J_{d}=15.0 Hz, Jₜ=6.5 Hz), 5.43-5.28 (12H, m), 5.18-5.12 (1H, m), 4.19-3.88 (6H, m), 3.53 (2H, brd, J=4.2 Hz), 3.21 (2H, brs), 2.87-2.79 (10H, m), 2.42-2.34 (4H, m), 2.11-1.99 (4H, m), 1.39-1.22 (26H, m), 0.97 (3H, t, J=7.6 Hz), 0.88 (3H, t, J=7.0 Hz).

### <Production of KIT-019>

Steps 1 to 11 are the same as those in the above “production of KIT-008”.

### Step 12: Introduction of Aliphatic Group (R²) at Sn-2 Position of Substrate

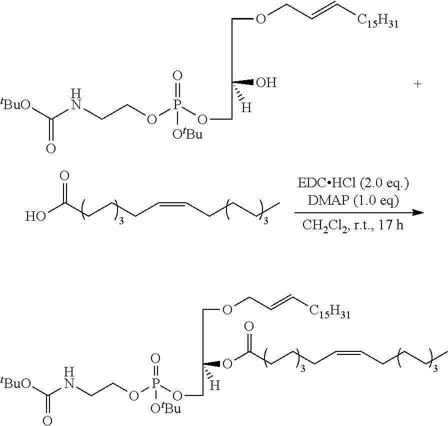

To a solution (0.5 mL) of tert-butyl (2-((tert-butoxy((R)-2-hydroxy-3-(((E)-octadec-2-en-1-yl)oxy)propoxy)phosphoryl)oxy)ethyl)carbamate (61.0 mg, 0.10 mmol) in dichloromethane was added a solution (0.5 mL) of 1-(3-dimethylaminopropyl)-3-ethylcarbodiimide hydrochloride (38.0 mg, 0.20 mmol) and dimethylaminopyridine (12.0 mg, 0.10 mmol)) and oleic acid (34.0 mg, 0.12 mmol) in dichloromethane at room temperature, and the mixture was stirred as it was for 17 hours. The reaction mixture was concentrated as it was under reduced pressure. The residue was purified by column chromatography to obtain the desired (2R)-1-((tert-butoxy(2-((tert-butoxycarbonyl)amino)ethoxy)phosphoryl)oxy)-3-(((E)-octadec-2-en-1-yl)oxy)propan-2-yl oleate as a colorless liquid (49.0 mg, 55%).

¹H NMR (500 MHz, CDCl₃) δ 5.68 (1H, dt, J=15.2 Hz, Jₜ=6.8 Hz), 5.49 (1H, dtt, J_{d}=15.3 Hz, Jₜ=1.3, 6.3 Hz), 5.38-5.30 (2H, m), 5.19-5.12 (2H, m), 4.21-4.15 (1H, m), 4.14-4.09 (1H, m), 4.08-4.01 (2H, m), 3.97-3.90 (2H, m), 3.55 (2H, d, J=5.2 Hz), 3.42-3.37 (2H, m), 2.33 (2H, t, J=7.6 Hz), 2.06-1.98 (6H, m), 1.65-1.58 (2H, m), 1.502 (9H, s, one diastereomer), 1.495 (9H, s, the other diastereomer), 1.44 (9H, s), 1.38-1.23 (46H, m), 0.88 (6H, t, J=6.9 Hz).

### Step 13: Deprotection and Amination at Sn-3 Position of Substrate

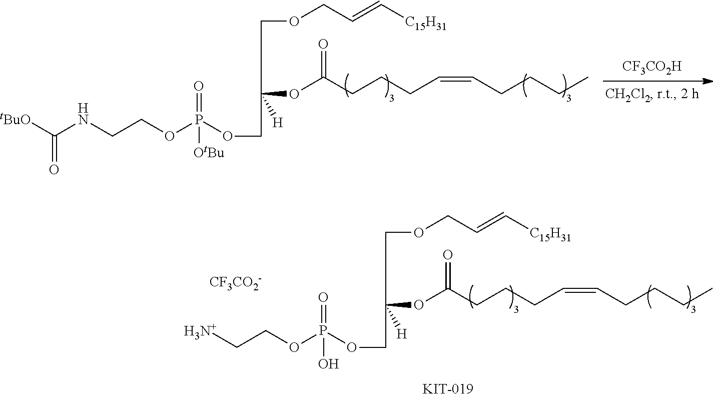

To a solution (0.55 mL) of (2R)-1-((tert-butoxy(2-((tert-butoxycarbonyl)amino)ethoxy)phosphoryl)oxy)-3-(((E)-octadec-2-en-1-yl)oxy)propan-2-yl oleate (46.0 mg, 0.055 mmol) in dichloromethane was added 0.55 mL of trifluoroacetic acid at room temperature, and the mixture was stirred as it was for 2 hours. The reaction mixture was concentrated as it was under reduced pressure to obtain the desired 2-((hydroxy((R)-3-3-(((E)-octadec-2-en-1-yl)oxy)-2-(oleoyloxy)propyl)phosphoryl)oxy)ethane-1-ammonium 2,2,2-trifluoroacetate (KIT-019) as a colorless liquid (46.2 mg, quant).

¹H NMR (500 MHz, CDCl₃) δ 7.88 (3H, brs), 5.68 (1H, dt, J_{d}=15.2 Hz, Jₜ=6.7 Hz), 5.48 (1H, dt, J_{d}=15.0 Hz, Jₜ=6.5 Hz), 5.38-5.30 (2H, m), 5.19-5.12 (1H, m), 4.22-3.97 (4H, m), 3.97-3.89 (2H, m), 3.53 (2H, brd, J=4.1 Hz), 2.32 (2H, t, J=7.6 Hz), 2.05-1.97 (6H, m), 1.61-1.55 (2H, m), 1.37-1.21 (m, 46H), 0.88 (6H, t, J=6.9 Hz).

### <Production of KIT-020>

Steps 1 to 11 are the same as those in the above “production of KIT-008”.

### Step 12: Introduction of Aliphatic Group (R²) at Sn-2 Position of Substrate

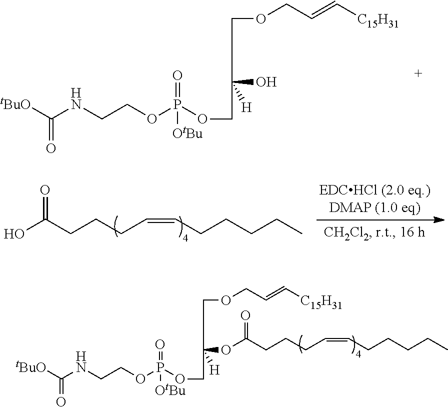

To a solution (0.5 mL) of tert-butyl (2-((tert-butoxy((R)-2-hydroxy-3-(((E)-octadec-2-en-1-yl)oxy)propoxy)phosphoryl)oxy)ethyl)carbamate (61.0 mg, 0.10 mmol) in dichloromethane was added a solution (0.5 mL) of 1-(3-dimethylaminopropyl)-3-ethylcarbodiimide hydrochloride (38.0 mg, 0.20 mmol) and dimethylaminopyridine (12.0 mg, 0.10 mmol) and arachidonic acid (37.0 mg, 0.12 mmol) in dichloromethane at room temperature, and the mixture was stirred as it was for 16 hours. The reaction mixture was concentrated as it was under reduced pressure. The residue was purified by column chromatography to obtain the desired (2R)-1-((tert-butoxy(2-((tert-butoxycarbonyl)amino)ethoxy)phosphoryl)oxy)-3-(((E)-octadec-2-en-1-yl)oxy)propan-2-yl(5Z,8Z,11Z,14Z)-icosa-5,8,11,14-tetraenoate as a colorless liquid (33.8 mg, 38%).

¹H NMR (500 MHz, CDCl₃) δ 5.68 (1H, dt, J=15.3 Hz, Jₜ=6.8 Hz), 5.49 (1H, dtt, J=15.3 Hz, Jₜ=1.3, 6.3 Hz), 5.43-5.30 (8H, m), 5.19-5.12 (2H, m), 4.21-4.16 (1H, m), 4.15-4.09 (1H, m), 4.08-4.01 (2H, m), 3.97-3.89 (2H, m), 3.55 (2H, d, J=5.2 Hz), 3.42-3.37 (2H, m), 2.86-2.78 (6H, m), 2.36 (2H, t, J=7.6 Hz), 2.11 (2H, dt, J_{d}=7.0 Hz, Jₜ=6.9 Hz), 2.06 (2H, dt, J_{d}=7.1 Hz, Jₜ=7.1 Hz), 2.03 (2H, dt, J_{d}=7.2 Hz, Jₜ=7.2 Hz), 1.75-1.67 (2H, m), 1.50 (9H, s, one diastereomer), 1.49 (9H, s, the other diastereomer), 1.44 (9H, s), 1.39-1.22 (32H, m), 0.89 (3H, t, J=6.9 Hz), 0.88 (3H, t, J=6.9 Hz).

### Step 13: Deprotection and Amination at Sn-3 Position of Substrate

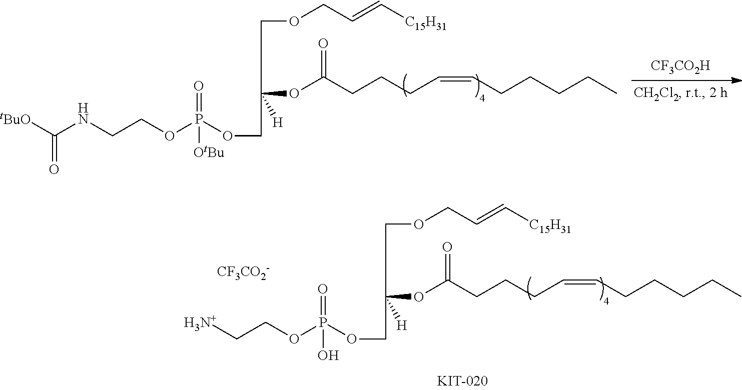

To a solution (0.36 mL) of (2R)-1-((tert-butoxy(2-((tert-butoxycarbonyl)amino)ethoxy)phosphoryl)oxy)-3-(((E)-octadec-2-en-1-yl)oxy)propan-2-yl(5Z,8Z,11Z,14Z)-icosa-5,8,11,14-tetraenoate (32.5 mg, 0.036 mmol) in dichloromethane was added 0.36 mL of trifluoroacetic acid at room temperature, and the mixture was stirred as it was for 2 hours. The reaction mixture was concentrated as it was under reduced pressure to obtain the desired 2-((hydroxy((R)-2-(((5Z,8Z,11Z,14Z)-icosa-5,8,11,14-tetraenoyl)oxy)-3-(((E)-octadec-2-en-1-yl)oxy)propoxy)phosphoryl)oxy)ethane-1-ammonium 2,2,2-trifluoroacetate (KIT-020) as a colorless liquid (31.3 mg, quant).

¹H NMR (500 MHz, CDCl₃) δ 7.91 (3H, brs), 5.68 (1H, dt, J_{d}=15.2 Hz, Jₜ=6.8 Hz), 5.48 (1H, dt, J_{d}=14.7 Hz, Jₜ=6.6 Hz), 5.43-5.30 (8H, m), 5.19-5.14 (1H, m), 4.22-4.12 (2H, m), 4.12-3.97 (2H, m), 3.96-3.89 (2H, m), 3.53 (2H, d, J=4.7 Hz), 3.30-3.21 (2H, m), 2.85-2.78 (6H, m), 2.35 (2H, t, J=7.7 Hz), 2.10 (2H, dt, J_{d}=7.2 Hz, Jₜ=7.2 Hz), 2.05 (2H, dt, J_{d}=7.5 Hz, Jₜ=7.5 Hz), 2.02 (2H, dt, J_{d}=7.4 Hz, Jₜ=7.4 Hz), 1.72-1.64 (2H, m), 1.38-1.22 (32H, m), 0.89 (3H, t, J=6.9 Hz), 0.88 (3H, t, J=7.5 Hz).

### Example 2

The effects of the compounds of the present invention produced above, KIT-008, KIT-019 and KIT-020 were confirmed.

### [Study 1: Evaluation of the IL-1β Reducing Effect (Anti-Inflammatory Effect) of the Compound of the Present Invention, KIT-008]

Mouse microglial cells (BV2) were cultured using a DMEM medium containing 2% FBS (fetal bovine serum) for 12 hours under conditions with or without the addition of the compound of the present invention, KIT-008 (5 μg/ml). Thereafter, the cells were treated with LPS (1 μg/ml) for 2 hours, and intracellular RNAs were extracted with an RNA extraction kit (TRI Reagent, Molecular Research Center Inc.), and the expression of an inflammatory cytokine IL-1β and the RNA expression of the Gapdh gene as a control were detected by semi-quantitative PCR (TaKaRa PrimeScript 1st strand cDNA synthesis Kit, TAKARA BIOTECHNOLOGY Co., LTD.).

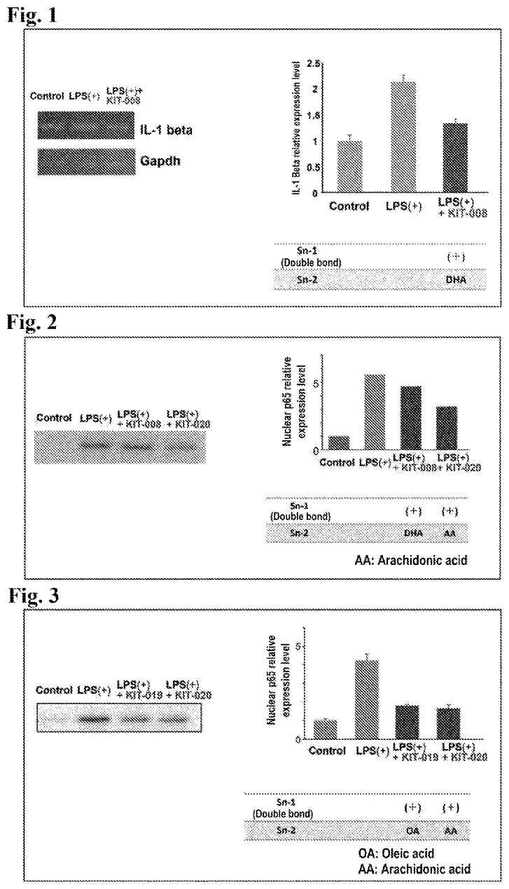

The results are shown in FIG. 1.

As shown in FIG. 1, the LPS-induced inflammatory cytokine IL-1β expression was reduced in the microglial cells (BV2) cultured in the medium with KIT-008 added.

### [Study 2: Evaluation of the p65 Nuclear Accumulation Suppression Effect (Anti-Inflammatory Effect) of the Compounds of the Present Invention, KIT-008 and KIT-020]

Mouse microglial cells (BV2) were cultured using a DMEM medium containing 2% FBS (fetal bovine serum) for 12 hours under conditions with or without the addition of the compound of the present invention, KIT-008 or KIT-020 (5 μg/ml). Thereafter, it was treated with LPS (1 μg/ml) for 2 hours, proteins in the cell nucleus were extracted, and p65 (NFkB) protein expression was detected by Western blotting using NF-κB p65 (Cell Signaling Technology, the clone name: D14E12) as a primary antibody and Anti-Rabbit IgG HRP-linked antibody (Cell Signaling Technology) as a secondary antibody.

The results are shown in FIG. 2.

As shown in FIG. 2, the LPS-induced p65 nuclear accumulation was suppressed in microglial cells (BV2) cultured in the medium with each of KIT-008 and KIT-020 added. In particular, it was revealed that the nuclear accumulation of p65 was significantly suppressed when KIT-020 was used.

### [Study 3: Evaluation of the p65 Nuclear Accumulation Suppression Effect (Anti-Inflammatory Effect) of the Compounds of the Present Invention, KIT-019 and KIT-020]

Mouse microglial cells (MG6) were cultured using a DMEM medium containing 2% FBS (fetal bovine serum) for 24 hours under conditions with or without the addition of the compound of the present invention, KIT-019 or KIT-020 (5 μg/ml). Thereafter, it was treated with LPS (1 μg/ml) for 8 hours, proteins in the cell nucleus were extracted, and p65 (NFkB) protein expression was detected by Western blotting using NF-κB p65 (Cell Signaling Technology, the clone name: D14E12) as a primary antibody and Anti-Rabbit IgG HRP-linked antibody (Cell Signaling Technology) as a secondary antibody.

The results are shown in FIG. 3.

As shown in FIG. 3, the LPS-induced p65 nuclear accumulation was significantly suppressed in microglial cells (MG6) cultured in the medium with each of KIT-019 and KRT20 added.

### [Study 4: Evaluation of the IL-1β Reducing Effect (Anti-Inflammatory Effect) of the Compounds of the Present Invention, KIT-019 and KIT-20]

Five-thousand mouse microglial cells (MG6) were cultured in a 96-well dish containing 100 μl of a DMEM medium containing 2% FBS (fetal bovine serum). Then, 5 μg/ml of each of the compounds of the present invention, KIT-019 and KIT-020 was added and cultured for 24 hours. Thereafter, it was treated with LPS (1 μg/ml) for 8 hours. The culture medium was collected, and the secreted IL-1β protein was measured by ELISA using DuoSet Mouse IL-1β (R&D Systems).

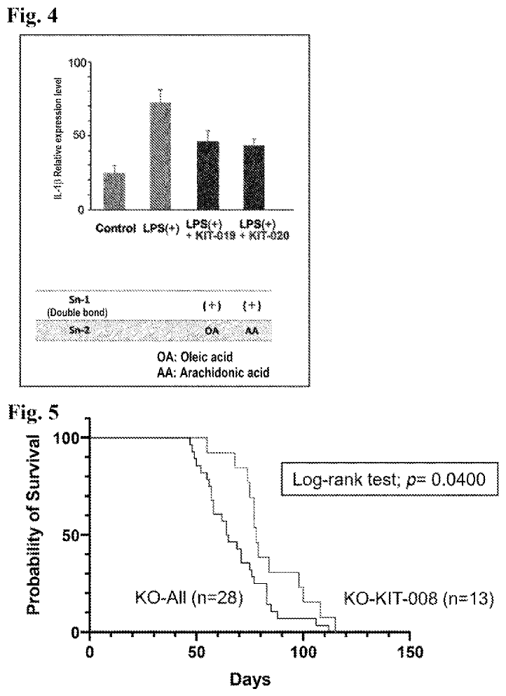

The results are shown in FIG. 4.

As shown in FIG. 4, the LPS-induced inflammatory cytokine IL-1β protein expression was significantly reduced in microglial cells (MG6) with each of KIT-019 and KIT-020 added.

The above shows that the compounds of the present invention exhibit an excellent anti-inflammatory effect.

### Example 3

Rett syndrome (RTT) is a progressive neurodevelopmental disorder caused by mutations in the MeCP2 gene located on the X chromosome. It is mainly developed in girls. Although the girls developing it grow normally for about 6 months to a year after birth, they thereafter exhibit various neurological symptoms such as a tendency towards autism. However, the details of the molecular mechanism by which MeCP2 dysfunction causes developmental disorders such as RTT have not been revealed.

In this Example, the influence of the compounds of the present invention on the growth of mice was investigated using MeCP2-deficient mice that exhibit a phenotype similar to that of RTT.

### [Study 1: Evaluation of Involvement in Lifetime]

Four-week-old male MeCP2-deficient mice were used in the experiment. The MeCP2-deficient mice were divided into two groups: a KIT-008 administration group (KO-KIT-008, n=13) and a control group (KO-All, n=28). The body weight of each of the mice in the administration group was measured in advance. The compound KIT-008 was suspended in water by sonication so that KIT-008 was ingested at 20 ng/g body weight, and it was continuously administered in drinking water to each mouse twice a week to verify its survival. The control group was given only water in the same manner.

The results are shown in FIG. 5.

As shown in FIG. 5, the MeCP2-deficient mice administered with the compound of the present invention, KIT-008 was higher in survival rate than the MeCP2-deficient mice not administered therewith. The number of days until the survival rate reached 50% was 64.5 days for mice in the control group. In contrast, the number of days until the survival rate reached 50% of mice in the KO-KIT-008 administration group was an average of 78 days.

The above shows that the mice administered with the compound of the present invention had a longer lifetime.

### [Study 2: Evaluation of RTT-Like Symptoms by Scoring]

MeCP2-deficient mice administered with each of the compounds of the present invention were evaluated for RTT-like symptoms by scoring. Four-week-old male MeCP2-deficient mice were used in the experiment. The MeCP2-deficient mice were divided into four groups: a KIT-020 administration group (KO-KIT-020, n=15), a KIT-019 administration group (KO-KIT-019, n=16), KIT-008 administration group (KO-KIT-008, n 13) and a control group (KO-2021, n=15). Each administration group was administered in drinking water with the compound that had been dissolved in water in the same manner as above so that the compound was ingested at 20 ng/g body weight. The control group was given only water in the same manner.

Scoring was performed once a week from 5 weeks of age on the following items 1 to 6.

### 1. Mobility

Each mouse was placed gently on a table and observed.

0 Point: The same mobility as that of the wild type.

1 Point: Less mobility than that of the wild type. (The freezing period is long when first placed on the table, and the time it remains still is long.)

2 Points: No spontaneous mobility after placed on the table. It can move in response to mild stimulation or food placed nearby.

### 2. Gait

0 Point: The same gait as that of the wild type.

1 Point: When walking or running, the distance between the hind legs is wider than that in the wild type, with a decrease in the height of the pelvis. Staggering gait. Waddling gait.

2 Points: More noticeable abnormality. Shaking when the legs are raised, walking backwards, lifting the hind legs at the same time.

### 3. Hindlimb Clasping

Each mouse was observed when it was hung by the base of its tail.

0 Point: Spreading its legs outward.

1 Point: Pulling the hind leg in the direction of the other leg or pulling one leg towards the body.

2 Points: Pulling the legs together tightly. The legs stick to each other or touch the body.

### 4. Tremor

Each mouse standing on a flat palm was observed.

0 Point: No shaking.

1 Point: Intermittent and mild shaking.

2 Points: Continuous shaking or intermittent severe shaking

### 5. Breathing

Flank movements were observed while the animal was in a stationary condition.

0 Point: Normal breathing.

1 Point: Both periods of regular breathing and faster breathing or breathing cessation for a short period of time are interspersed.

2 Points: Very irregular breathing. Panting or shortness of breath.

### 6. General Condition

Observations were made based on general well-being indicators such as coat condition, eyes and posture.

0 Point: Beautiful and shiny coat, clean eyes, normal posture.

1 Point: Blank eyes, lackluster coat/no grooming, slightly hunched posture.

2 Points: Eyes closed or squinted, piloerection, hunched posture.

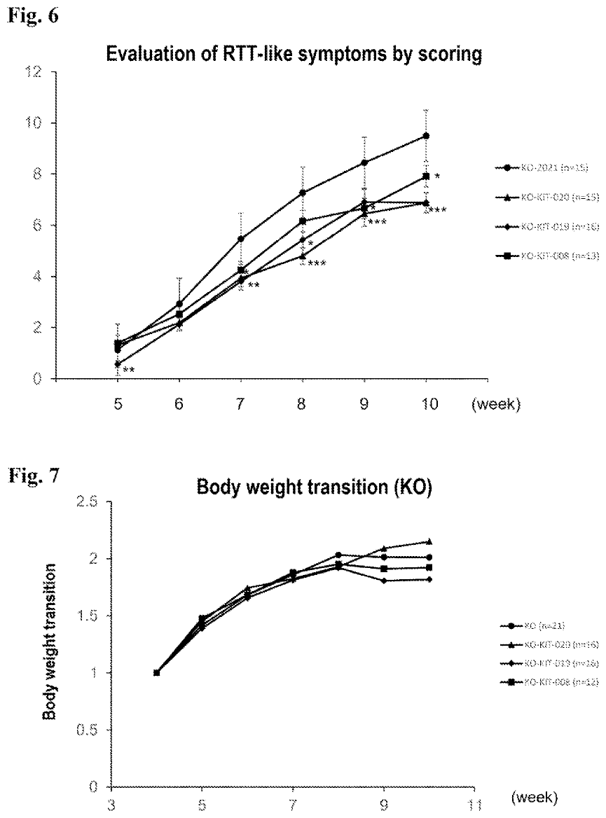

The results are shown in FIG. 6.

As shown in FIG. 6, it was confirmed that the score of RTT-like symptoms of mice was reduced by using each of the compounds of the present invention. Therefore, it was revealed that RTT-like symptoms were ameliorated by administering each of the compounds of the present invention.

### [Study 3: Evaluation of Body Weight Transition]

The body weight of each of MeCP2-deficient mice administered with the compound of the present invention was measured every week, and the body weight transition was evaluated. Four-week-old male MeCP2-deficient mice were used in the experiment. The MeCP2-deficient mice were divided into four groups: a KIT-020 administration group (KO-KIT-020, n=16), a KIT-019 administration group (KO-KIT-019, n=16), KIT-008 administration group (KO-KIT-008, n=12) and a control group (KO-2021, n=21). Each administration group was administered in drinking water with the compound that had been dissolved in water in the same manner as above so that the compound was ingested at 20 ng/g body weight. The control group was given only water in the same manner.

The results are shown in FIG. 7. The body weight transition is expressed as a relative value to the initial body weight.

As shown in FIG. 7, no statistical difference was found between the body weight transition of the mice administered with the compound of the present invention and the body weight transition of KO mice in the control group (t-test).

### [Study 4: Evaluation of Toxicity]

The toxicity of the compounds of the present invention to mice was evaluated based on the body weight transition of wild-type mice administered with each of the compounds of the present invention.

20 ng/g body weight of each of the compounds of the present invention was administered in drinking water to 4-week-old male wild-type mice in the same manner as that in Study 3. The control group of the wild-type mice was given only water.

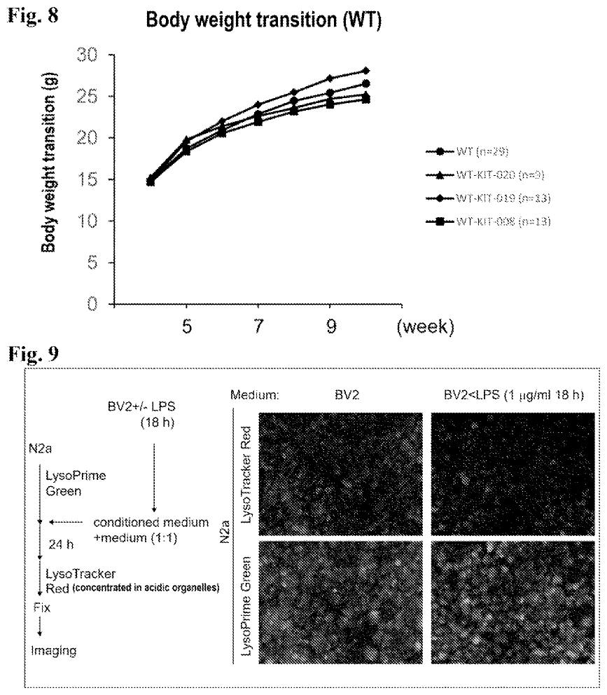

The results are shown in FIG. 8.

As shown in FIG. 8, weight gain similar to that of the control group was confirmed in the mice administered with each of the compounds of the present invention.

Therefore, the compounds of the present invention are considered to be nontoxic.

### Example 4

“Lysosome” which is one of organelles contains enzymes that work under acidic conditions and are involved in the decomposition, removal and recycling of metabolic wastes, proteins and nucleic acids, and the like. However, when neurons are damaged by diseases such as Alzheimer's disease, the acidic level within lysosomes decreases, and the lysosomes enlarge as they combine with “autophagic vacuoles” filled with undecomposed waste products. These autophagic vacuoles also contain early amyloid beta (Aβ), and before amyloid beta accumulates, neurons are destroyed due to impaired acidity within the neuronal lysosomes (Ju-Hyun Lee et al. (2022) Faulty autolysosome acidification in Alzheimer's disease mouse models induces autophagic build-up of Aβ in neurons, yielding senile plaques. Nat. Neurosci. 25: 688-701).

### [Study 1]

In this study, the influence of the compounds of the present invention on impaired lysosomal acidity was investigated.

[Preliminary Study: Establishment of an Experimental System that Impairs Lysosomal Acidity]

Lysosomes in mouse neurons Neuro2a (N2a) cultured in a normal DMEM medium were labeled with LysoPrime Green (Dojindo). LysoPrime Green is a fluorescent dye that is highly specific for lysosomes which are acidic organelles and that is resistant to pH changes.

Then, N2a is cultured in the culture supernatant of mouse glial cells BV2 treated with the endotoxin lipopolysaccharide (LPS) (1 μg/ml, 18 h) or the culture supernatant of untreated BV2, and labeled with LysoTracker™ Red DND-99 (Thermo Fisher Scientific) which targets acidic organelles.

As a result, as shown in FIG. 9, targeting, by of LysoTracker Red, of the lysosomes of N2a cultured in the culture supernatant of the BV2 administered with LPS was significantly suppressed, and the lysosomal acidity was observed to be impaired. That is, it was shown that acidification of the lysosomes in the neurons were impaired by glial cell-derived factors in the medium resulting from inflammation.

### [Study: Confirmation of the Effect of the Compounds of the Present Invention on Impaired Lysosomal Acidity]

The effect of each of the compounds of the present invention on the impaired lysosomal acidification was confirmed using the above experimental system.

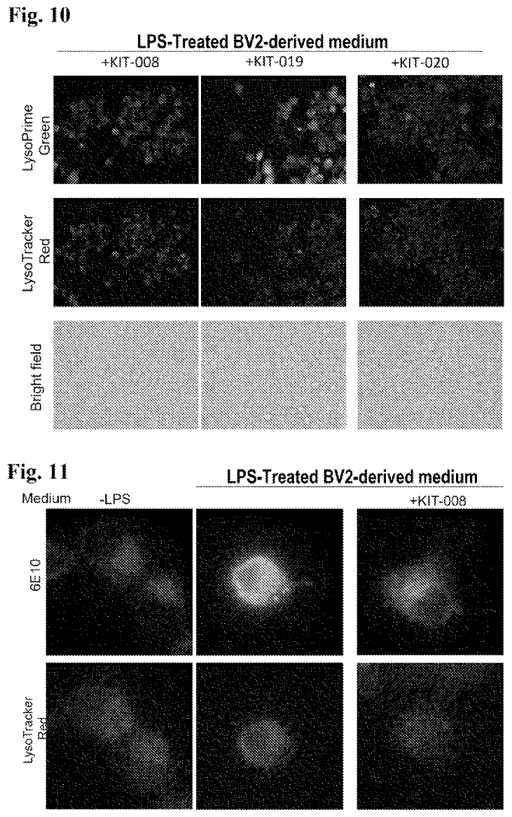

Each of the compounds of the present invention, KIT-008, KIT-019 and KIT-020 was added to the medium of N2a cells and cultured for 24 hours, and then cultured for another 24 hours in the culture supernatant of BV2 administered with LPS in the presence of each compound. As a result, as shown in FIG. 10, the compounds of the present invention, KIT-008, KIT-019, and KIT-020 ameliorated the impaired acidification of lysosomes in neurons caused by glial cell-derived factors. Among them, KIT-008 had the highest effect.

### [Study 2]

In this study, it was investigated whether the compound of the present invention actually suppresses amyloid beta (Aβ) accumulation in N2a cells.

A plasmid having cDNA encoding CFPAPP_{sw} (CFPAPP_{sw} that fuses a fluorescent protein CFP to the N-terminus of an amyloid precursor protein (APP_{sw}) having a Swedish-type amino acid substitution that is easily cleaved by BACE) was expressed in N2a cells, and localization of amyloid beta (Aβ) in the N2a cells in an untreated mouse glial cell BV2 culture supernatant (−LPS), in the N2a cells in LPS-treated BV2 culture supernatant (LPS-treated BV2-derived medium), and in the N2a cells administered with KIT-008 was verified using an antibody 6E10 (BioLegend) that recognizes the Aβ portion.

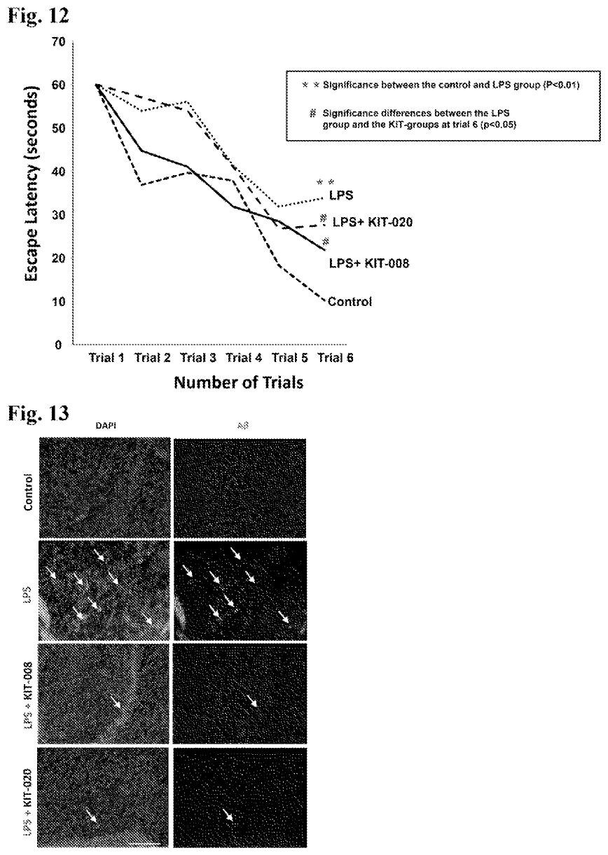

As a result, as shown in FIG. 11, Aβ was not localized in lysosomes in the N2a cells in normal medium, but Aβ was detected in lysosomes in the N2a cells in which lysosomal acidification had been impaired due to glial cell-derived factors resulting from inflammation. This accumulation of Aβ in lysosomes was eliminated by pretreatment with KIT-008. That is, it is believed that the accumulation of Aβ in lysosomes due to impaired lysosomal acidity, which is observed in the early stages of the onset of Alzheimer's disease, was suppressed by amelioration of the impaired lysosomal acidity caused by KIT-008.

The compound of the present invention ameliorates the impaired lysosomal acidity and suppresses the accumulation of amyloid beta (Aβ) in lysosomes, and therefore are expected to be effective in preventing and ameliorating Alzheimer's disease.

### Example 5

The influence of the compounds of the present invention on LPS-induced memory impairment in mice was investigated.

Specifically, 8-week-old male c57BL6J mice (5 mice in each group, n=5) were allowed to drink each of the compounds of the present invention (KIT-008 and KIT-020) at a dose of 10 mg/50 kg/day for 4 weeks, and were then subjected to LPS treatment (200 mg/kg/day) for 7 days while allowing them to drink the compound in the same manner. Thereafter, they were subjected to a water maze test for 2 days (3 trials per day).

The results are shown in FIG. 12. Escape Latency indicates the time required to arrive at an escape platform (arrival time). Comparisons between groups were performed by ANOVA test and Bonferroni's post hoc test. The LPS group showed decreased memory retention (increased arrival time) compared to the control group (P<0.01). In Trial 6, the KIT-008 group and the KIT-020 group showed improvement in memory retention compared to the LPS group (P<0.05 for each). Therefore, the compounds of the present invention are effective in improving brain function (cognitive function).

### Example 6

The influence of compounds of the present invention on LPS-induced amyloid beta (Aβ) deposition in mouse brain tissue (hippocampal tissue) was investigated.

Specifically, 8-week-old male c57BL6J mice were allowed to drink each of the compounds of the present invention (KIT-008 and KIT-020) at a dose of 10 mg/50 kg/day for 4 weeks, and were then subjected to LPS treatment (200 mg/kg/day) for 10 days while allowing them to drink the compound in the same manner. The mouse hippocampal tissue was subjected to an immunohistochemistry test using an anti-amyloid beta antibody (a cocktail of two antibodies, 6E10 (BioLegend) and 82E1 (Immuno-Biological Laboratories Co, Ltd)). DAPI was used to stain a cell nucleus.

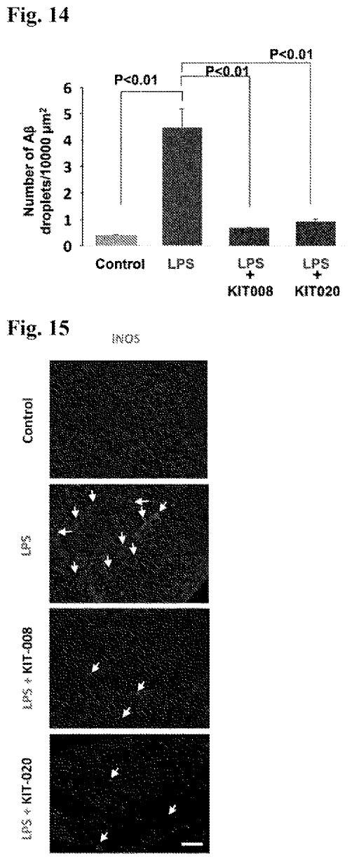

The results are shown in FIG. 13 and FIG. 14 (graph). In FIG. 13, the scale bar is 100 μm and the arrows indicate amyloid beta that deposited. The data in FIG. 14 represent the average number of amyloid beta deposition in each group (3 mice, n=3). In the LPS group, amyloid beta deposition was observed compared to the control group, but in the KIT-008 group and the KIT-020 group, amyloid beta deposition caused by LPS was suppressed.

### Example 7

The influence of the compounds of the present invention on induced expression of inducible nitric oxide synthase (iNOS) was investigated, using increased expression of iNOS caused by intracerebral inflammation in mouse brain tissue (hippocampal tissue) as an indicator.

Specifically, 8-week-old male c57BL6J mice were allowed to drink each of the compounds of the present invention (KIT-008 and KIT-020) at a dose of 10 mg/50 kg/day for 4 weeks, and were then subjected to LPS treatment (200 mg/kg/day) for 10 days while allowing them to drink the compound in the same manner. The mouse brain tissue was subjected to an immunohistochemistry (IHC) study using an anti-NeuN antibody (a neuron marker, Millipore MAB377) and an anti-iNOS antibody (an inflammatory marker, Invitrogen PA1-036). DAPI was used to stain a cell nucleus.

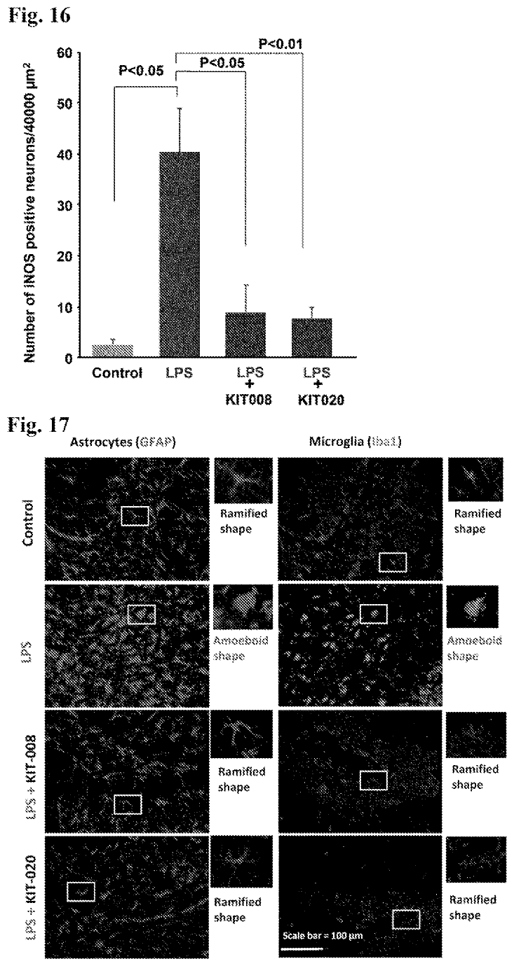

The results are shown in FIG. 15 and FIG. 16 (graph). In FIG. 15, the scale bar is 50 μm and the arrows indicate iNOS-positive neurons. The data in FIG. 16 represents the average value for each group (3 mice, n=3). In the LPS group, the number of iNOS-positive neurons increased compared to that in the control group, but in the KIT-008 group and the KIT-020 group, the increase in iNOS-positive neurons caused by LPS was suppressed.

### Example 8

The influence of the compounds of the present invention on LPS-induced amoeboid shaped microglia (Iba1-positive cells) increase and amoeboid shaped astrocytes (GFAP-positive cells) increase in mouse brain tissue (hippocampal tissue) were investigated. The amoeboid shape represents inflammation in microglia and astrocytes. The influence of the compounds of the present invention on LPS-induced astrocytes (GFAP-positive cells) increase in mouse brain tissue (hippocampal tissue) was also investigated.

Specifically, 8-week-old male c57BL6J mice were allowed to drink each of the compounds of the present invention (KIT-008 and KIT-020) at a dose of 10 mg/50 kg/day for 4 weeks, and were then subjected to LPS treatment (200 mg/kg/day) for 10 days while allowing them to drink the compound in the same manner. The mouse brain tissue was subjected to an immunohistochemistry (IHC) study using Iba1 (a microglia marker, Fujifilm 019-19741) and GFAP (an astrocyte marker, Sigma C9205). DAPI was used to stain a cell nucleus.

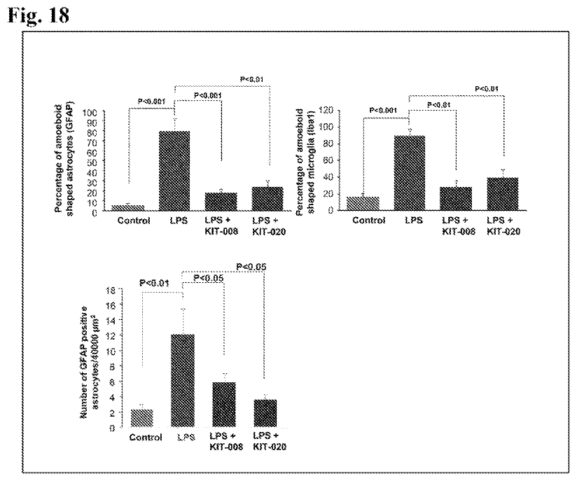

The results are shown in FIG. 17 and FIG. 18 (graph). In FIG. 17, the scale bar is 100 μm. The data in FIG. 18 represent the percentages of amoeboid shaped microglia and amoeboid shaped astrocytes (upper graphs) and the number of astrocytes (GFAP-positive cells) (a lower graph) in each group (3 mice, n=3). In the LPS group, the amoeboid shaped microglia (Iba1-positive cells) and the amoeboid shaped astrocytes (GFAP-positive cells) increased compared to the control group. In addition, the astrocytes (GFAP-positive cells) increased. However, in the KIT-008 group and the KIT-020 group, the LPS-induced amoeboid shaped microglia (Iba1-positive cells) increase and amoeboid shaped astrocytes (GFAP-positive cells) increase and astrocytes (GFAP-positive cells) increase were suppressed.

### INDUSTRIAL APPLICABILITY

The novel compound of the present invention can be used as a pharmaceutical composition and the like, and is therefore industrially useful.

1. A compound represented by the general formula (I) or its racemic form, or their salt: 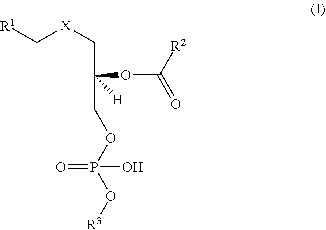
wherein X represents an oxygen atom, a nitrogen atom or a sulfur atom; R¹ represents an unsaturated aliphatic hydrocarbon group; R² represents a saturated or unsaturated aliphatic hydrocarbon group; and R³ represents choline, ethanolamine, inositol or serine.

2. The compound or its racemic form, or their salt according to claim 1, wherein X is an oxygen atom.

3. The compound or its racemic form, or their salt according to claim 1, wherein R¹ has at least one double bond.

4. The compound or its racemic form, or their salt according to claim 3, wherein R¹ has one double bond.

5. The compound its racemic form, or their salt according to claim 4, wherein the double bond of R¹ is present between the carbon at position 1 and the carbon at position 2, when the carbon bonded to the carbon bonded to X is defined as the carbon at position 1.

6. A composition comprising the compound, its racemic form, or their salt according to claim 1 as an active ingredient.

7. The composition according to claim 6, wherein the composition has an anti-inflammatory effect.

8. The composition according to claim 6, wherein the composition is for preventing or ameliorating cranial nerve inflammatory diseases.

9. The composition according to claim 8, wherein the cranial nerve inflammatory diseases are at least one disease selected from dementia, Parkinson disease, depression and schizophrenia.

10. The composition according to claim 6, wherein the composition is for preventing or ameliorating Rett syndrome.

11. The composition according to claim 6, wherein the composition is a pharmaceutical composition.
